(19) United States
(12) Patent Application Publication
(10) Pub. No.: US 2005/0244853 A1
(43) Pub. Date: Nov. 3, 2005

(54) Determining relative expression for probe groups in probe arrays

(75) Inventors: James Bradley Aimone, San Diego, CA (US); Fred H. Gage, La Jolla, CA (US)

Correspondence Address: MORRISON & FOERSTER LLP, 425 MARKET STREET, SAN FRANCISCO, CA, 94105-2482, US

(73) Assignee: The Salk Institute For Biological Studies, La Jolla, CA (US)

(21) Appl. No.: 11/043,278

(22) Filed: Jan. 25, 2005

Related U.S. Application Data
(60) Provisional application No. 60/539,907, filed on Jan. 27, 2004.

(51) Int. Cl. C12Q 1/68; G06F 19/00; G01N 33/48; G01N 33/50

(52) U.S. Cl. 435/6; 702/20

(57) ABSTRACT

A method of determining relative expression for probe groups in probe arrays includes: determining probe values for one or more probe arrays of a baseline category and multiple probe arrays of an experimental category, where each probe array includes a plurality of probes organized by probe locations; determining, from the probe values corresponding to the probe locations, probe categories for the probe locations, where each probe category corresponds to the baseline category, the experimental category or an indefinite category; and determining, from the probe values and the probe categories, a category bias for a probe group, where the probe group includes multiple probe locations, and the category bias including a preference for the baseline category, the experimental category or the indefinite category.

### CROSS-REFERENCE TO RELATED APPLICATIONS

This application claims the benefit of provisional application 60/539,907, filed Jan. 27, 2004, and incorporated herein in its entirety by reference.

### STATEMENT REGARDING FEDERALLY SPONSORED RESEARCH OR DEVELOPMENT

This invention was made with support under grant number R01 AG021876 from the National Institutes of Health. The Government may have rights to this invention.

### BACKGROUND OF THE INVENTION

The present invention relates to genomics generally and more particularly to the analysis of genomic probe arrays.

Genomic probe arrays such as Affymetrix GeneChips are being used increasingly for quantitative monitoring of gene expression in a variety of biological systems. Depending on the experiment, the analysis of these results can have several different goals ranging from calculation of signal strength for a variety of inter-gene comparisons to the determination of which genes show significant differential expression between sample conditions. There have been several proposed methods for precise quantification of expression signal with promising results, however the question of what constitutes a significant change between replicate groups still remains.

The use of large-scale screening of mRNA expression has increased in the past several years. Since the introduction of a high-density system in 1996 by Affymetrix, there have been considerable improvements in experimental protocols and product quality. However, biologists have struggled conceptually to understand the biological relevance of mRNA assays and the analysis and interpretation has presented far more of a challenge to biologists than the merits of the underlying tool itself. (Lockhart, D. J., Dong, H., Byrne, M. C., Follettie, M. T., Gallo, M. V., Chee, M. S., et al., Expression monitoring by hybridization to high-density oligonucleotide arrays, Nat Biotechnol 1996; 14: 1675-80.)

Affymetrix GeneChips have been used for many different assays including drug efficacy screening, temporal profiles, and tissue-specific screens. (Hu, J. S., Durst, M., Kerb, R, Truong, V., Ma, J. T., Khurgin, E., et al., Analysis of drug pharmacology towards predicting drug behavior by expression profiling using high-density oligonucleotide arrays, Ann N Y Acad Sci 2000; 919: 9-15; Cavallaro, S, D'Agata, V., Manickam, P., Dufour, F., Alkon, D. L., Memory-specific temporal profiles of gene expression in the hippocampus, Proc Natl Acad Sci USA 2002; 99: 16279-84; Zhao, X., Lein, E. S., He, A., Smith, S. C., Aston, C., Gage, F. H., Transcriptional profiling reveals strict boundaries between hippocampal subregions, J Comp Neurol 2001; 441: 187-96.)

Recently these GeneChips have been used increasingly in the global characterization of biological systems—experiments whose magnitude makes gene-by-gene validation difficult. Initially, many of these large experiments were accompanied by verification of resultant genes by RT-PCR and in situ hybridizations. (Lockhart, D. J., Barlow, C., Expressing what's on your mind: DNA arrays and the brain., Nat Rev Neurosci 2001; 2: 63-8; Sandberg, R., Yasuda, R., Pankratz, D. G., Carter, T. A., Del Rio, J. A., Wodicka, L., et al., Regional and strain-specific gene expression mapping in the adult mouse brain, Proc Natl Acad Sci USA 2000; 97: 11038-43.)

However, several recent studies have included little or no follow-up validation. (Ivanova, N. B., Dimos, J. T., Schaniel, C., Hackney, J. A., Moore, K. A., Lemischka, I. R., A stem cell molecular signature, Science 2002; 298: 601-4; Ramalho-Santos, M., Yoon S., Matsuzaki, Y., Mulligan, R. C., Melton, D. A., “Stemness”: transcriptional profiling of embryonic and adult stem cells, Science 2002; 298: 597-600.).

Although there may be no uniform standard for biological significance in a system, a more apparent need emerges for a uniform standard for statistical significance, especially if results from these experiments are going to be considered an end-result. There are several proven techniques used for initial analysis including: MAS 5.0, dChip, Naef's method, and RMA. However, these techniques often provide significantly different results. Furthermore, determination of differentially expressed genes is also inconsistent, often resulting in the biologist making empirical decisions such as fold change cut-offs. These assumptions may become important if unverified results are published as definitive results. (Hubbell, E., Liu, W. M., Mei, R., Robust estimators for expression analysis, Bioinformatics 2002; 18: 1585-92; Liu, W. M., Mei, R., Di, X., Ryder, T. B., Hubbell, E., Dee, S., et al., Analysis of high density expression microarrays with signed-rank call algorithms, Bioinformatics 2002; 18: 1593-9, Li, C., Wong, W. H., Model-based analysis of oligonucleotide arrays: expression index computation and outlier detection, Proc Natl Acad Sci USA 2001; 98: 31-6; Naef, F., Hacker, C. R., Patil, N., Magnasco, M., Empirical characterization of the expression ratio noise structure in high-density oligonucleotide arrays, Genome Biol 2002; 3; Irizarry, R. A., Hobbs, B., Collin, F., Beazer-Barclay, Y. D., Antonellis, K. J., Scherf, U., et al., Exploration, normalization, and summaries of high density oligonucleotide array probe level data, Biostatistics 2003; 4: 249-64.)

Analysis at the probe level should be carried out in using statistical methods that are consistent at the probe level and furthermore support higher-level analysis for determining a corresponding expression for a probe group (e.g., a “gene list”). If “gene lists” are going to be published, then they should be compiled using consistent statistical techniques, a requirement that is often compromised by assumptions regarding outliers and systemic variance. Statistically, a change is only significant if it meets certain criteria in spite of outliers and noise. Determination of which genes change significantly (e.g., in terms of expression) should be made independently from the analytical quantification of expression level and biological filtering at the probe level.

Thus, there is a need for an improved determination of expression for probe groups in probe arrays.

### SUMMARY OF THE INVENTION

In one embodiment of the present invention, a method of determining relative expression for probe groups in probe arrays includes: determining probe values for one or more probe arrays of a baseline category and multiple probe arrays of an experimental category, where each probe array includes a plurality of probes organized by probe locations; determining, from the probe values corresponding to the probe locations, probe categories for the probe locations, where each probe category corresponds to the baseline category, the experimental category or an indefinite category; and determining, from the probe values and the probe categories, a category bias for a probe group, where the probe group includes multiple probe locations, and the category bias including a preference for the baseline category, the experimental category or the indefinite category.

According to one aspect of this embodiment, the one or more probe arrays of the baseline category may include a plurality of probe arrays.

According to another aspect of this embodiment, determining probe values may include: measuring the probe values from expression levels in the probe arrays; and adjusting the probe values to remove background signals.

According to another aspect of this embodiment, determining probe categories for the probe locations may include: determining probe ratios from the probe values at probe locations for combinations of the baseline-category arrays and the experimental-category arrays; normalizing the probe ratios at the probe locations; and determining, from the normalized probe ratios at the probe locations, probe categories for the probe locations. This aspect may further include: changing the probe categories at one or more probe locations by determining and comparing confidence values for the baseline category, the experimental category, and the indefinite category at the one or more probe locations.

According to another aspect of this embodiment, determining the category bias for the probe group may include: determining probe uncertainties for the probe locations in the probe group; and determining, from the probe categories and probe uncertainties for the probe locations in the probe group, the category bias and a confidence for the category bias. According to this aspect, determining the category bias and the confidence for the category bias may include: performing a signed rank test on an ordering of the probe categories according to the probe uncertainties.

Additional embodiments relate to an apparatus that carries out the method and computer-readable media where the method is stored as a computer program. In this way, the present invention enables an improved determination of expression for probe groups in probe arrays.

### BRIEF DESCRIPTION OF THE DRAWINGS

FIG. 1 shows a probe array as used by an embodiment of the present invention.

FIG. 2 shows probe arrays corresponding to a baseline category and an experimental category as used by an embodiment of the present invention.

FIG. 3 shows a method of determining relative expression for probe groups in probe arrays according to an embodiment of the present invention.

FIG. 4 shows further detail for the embodiment shown in FIG. 3.

FIG. 5 shows further detail for the embodiment shown in FIG. 3.

FIG. 6 shows further detail for the embodiment shown in FIG. 3.

FIGS. 7A-7C show further detail for the embodiment shown in FIG. 3.

FIG. 8 shows further detail for the embodiment shown in FIG. 3.

FIGS. 9A-9B show results for an embodiment of the present invention as applied to spike-in data.

FIGS. 10A-10D show results for an embodiment of the present invention as applied to laboratory data.

FIG. 11 shows a table that summarizes results for the embodiment shown in FIGS. 10A-10D.

### DETAILED DESCRIPTION OF EXEMPLARY EMBODIMENTS

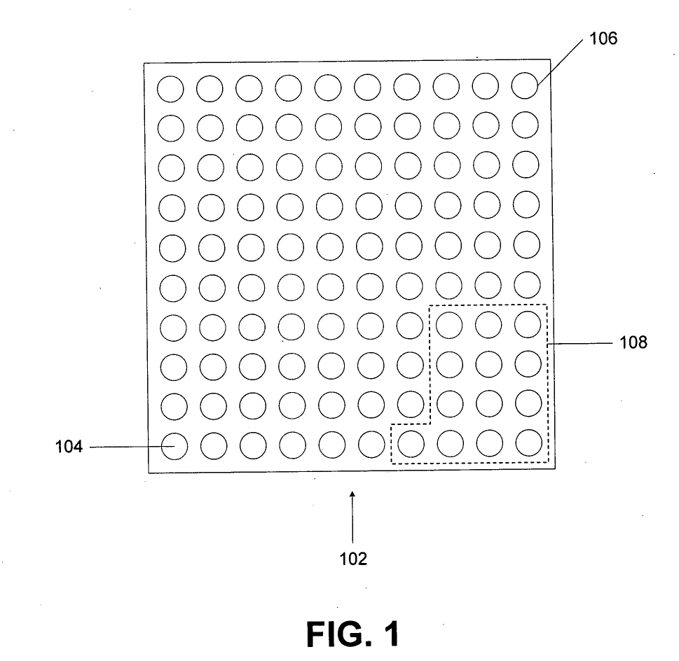

FIG. 1 shows a probe array 102 as used by an embodiment of the present invention. The array 102 has one hundred probes arranged uniformly in ten rows and ten columns. Individual probes can be indexed according to probe location in the array 102 so that for example the (1,1)-probe 104 is in the lower left corner of the array 102 and the (10,10)-probe 106 is in the upper right corner of the array 102. A probe group 108 of probe locations includes thirteen elements that correspond to a specific gene sequence.

The dimensions shown in FIG. 1 are convenient for illustrating the present invention. In applications involving Affymetrix GeneChips, most probe arrays contain between two-hundred thousand and six-hundred thousand probes (some potentially over one million probes). Typically, a probe group includes eleven to twenty probes (with distinct probe locations), where the individual probes independently measure different coding regions of the same target gene. Depending on the array type, there may be five thousand to over forty thousand of such genes (or probe groups) measured in this way on a single chip.

Probe arrays can be used to assess the relative expression for a probe group as a comparison between two categories (or sources) of probe data. For example, a specific cell type (e.g., brain cells, liver cells) may be designated as a category and used to generate an array of probe data (e.g., mRNA expression) by exposing a probe array 102 to a solution of mRNA from cells of that cell type. Each array of probe data may be considered as a completed experiment for that cell type.

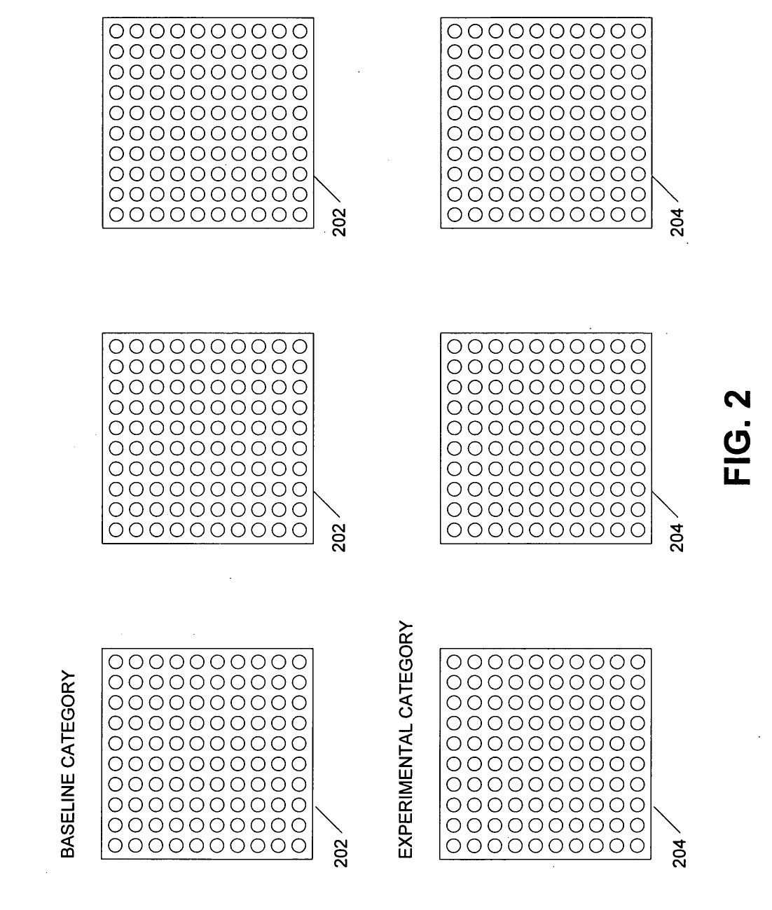

For example, FIG. 2 shows three arrays 202 of probe data of a baseline (B) category and three arrays 204 of probe data of an experimental (E) category. Preferably at least two to four arrays for each category are used; however, any number of B-category arrays and any number of E-category arrays may be considered as long as there are multiple arrays in at least one category so that some statistical significance is obtained. For example, the method may be applied to a single array of the baseline (B) category and multiple arrays of the experimental (E) category. It should be noted that these particular designations (baseline, experimental) are arbitrary labels that can be applied to any comparison between to two categories. An array of probe data of a specific category may also be denoted as a probe array of that category.

Based on probe data, one may ask whether the given probe group 108 is more strongly expressed in the B-category arrays 202 or the E-category arrays 204. For the case where the B-category denotes brain cells, the E-category denotes liver cells, and probe group denotes gene X, this corresponds to determining whether gene X is more strongly expressed in brain cells or liver cells.

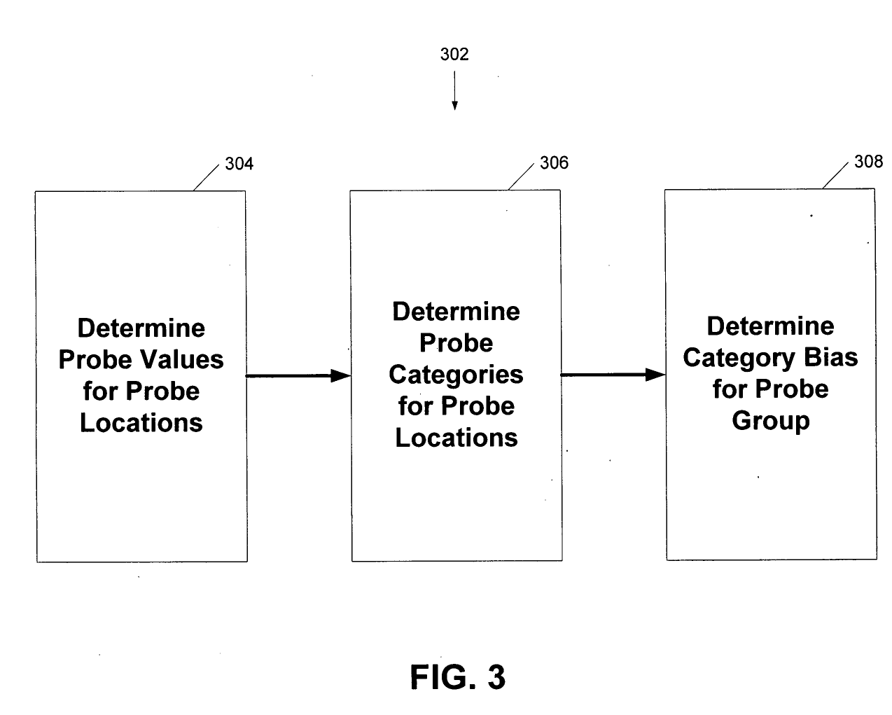

FIG. 3 shows a method 302 of determining relative expression for probe groups in probe arrays according to an embodiment of the present invention. First, probe values are determined 304 at probe locations. With reference to FIG. 1, each probe array 102 results in one-hundred probe values for the one-hundred probe locations. With reference to FIG. 2, each of the B-chips 202 results in one-hundred probe values and each of the E-chips 204 results in one-hundred probe values. For the example shown, the given probe group 108 specifies thirteen probe locations from the one-hundred possible locations. Next probe categories are determined 306 for the probe locations. With reference to FIG. 1, each of the probe locations (e.g., as indexed from (1,1) to (10,10)) is assigned a category value of “baseline”, “experimental” or “indefinite” depending on whether the probe values show a statistical bias towards one of underlying categories (or to neither). Next an overall category bias is determined 308 for the probe group 108 including a preference for the baseline category, the experimental category or the indefinite category. In the above example involving brain cells and liver cells, the category bias indicates whether gene X is more strongly expressed in brain cells or liver cells or neither.

Although the above discussion describes determining probe values 304 and determining probe categories 306 for all probe locations that are physically on the arrays 102, it is possible to restrict the analysis to a smaller set of probe locations that are sufficient for the carrying out the statistically-based operations described below (e.g., for providing a sufficient confidence level). That is, some of the probe locations on the arrays 102 may be ignored in the analysis so that the term “probe locations” refers to a smaller set of relevant probe locations. However, in many operational settings it is preferable to determine probe values and probe categories for substantially all available probe locations so that the results can be applied flexibly to arbitrarily defined probe groups with greater statistical confidence.

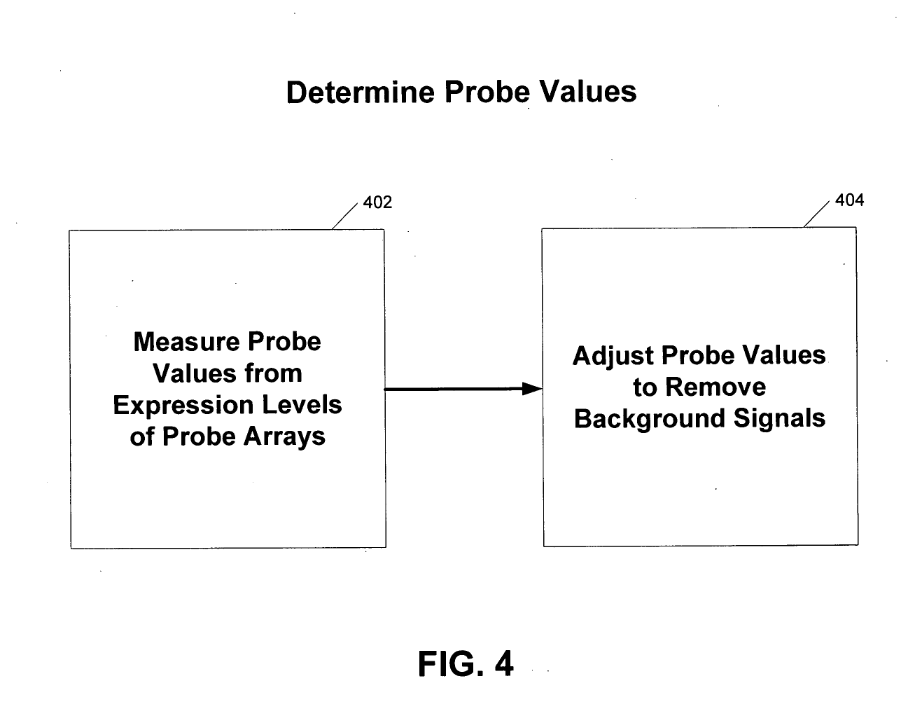

FIG. 4 shows further detail for determining probe values 304 for the probe locations. First, probe values are measured 402 at the probe locations from probe data (e.g., expression levels) in the probe arrays. Typically each probe is a small blot of millions of identical 25-base oligonucleotides fixed to the chip. Fluorescence measurements of hybridized tagged mRNA are used to assess the mRNA expression level of that sequence. A common form for this measurement is a digitized image file (e.g., a *.cel file).

Next probe values are adjusted 404 to remove background signals. Background signals can come in several forms in these experiments. On a global scale, there is noise that affects the entire experiment, such luminescence and scanner effects, which essentially shifts the expression levels of every probe on the chip by a roughly equivalent amount. While these general biases are only slightly probe dependent, there are more sequence specific noise effects which affect some probes more than others since some sequences are more susceptible to non-specific hybridization than others. Both of these noise contributions have a large additive component which is relatively independent of the concentration of the target sequence. Therefore, as a preliminary step, an estimate of the additive component of non-specific binding and fluorescence for each probe is subtracted from the corresponding measured value at that probe.

The Affymetrix GeneChip system was designed with this subtraction in mind. Each 25-base target probe (Perfect Match, PM) is adjacent to a near identical probe (Mismatch, MM) with the exception of the 13th base—which is changed to minimize specific binding. The theory of these MM probes is that their sequence is close enough to the PM probe to have similar specific noise contributions and their proximal location on the chip gives them similar global background contributions, but because of the change of the middle base they have a greatly reduced “target” signal. Therefore, the original Affymetrix plan was that the MM probe would serve as an estimation of background signal for the PM probe, thus making the PM-MM subtraction the most accurate measurement of the concentration of the target oligonucleotide. (Affymetrix. Affymetrix Microarray Suite User Guide. Version 4.0. Affymetrix: Santa Clara, Calif., 2000.)

However, the MM probe subtraction has been criticized by some, primarily because the MM probes seem to have erratic binding to the true target sequence. This can result in MM probes with intensities similar to or even higher than their PM counterparts. The subtraction of an inconsistent amount of real signal from the PM probes would be problematic, and has led many to tailor their analysis to look at the PM probes only, with more sophisticated background subtraction techniques. These methods often account for probe-specific noise contributions by using ratio measures and modeling across the probe set. (Naef, F., Hacker, C. R., Patil, N., Magnasco, M., Empirical characterization of the expression ratio noise structure in high-density oligonucleotide arrays, Genome Biol 2002: 3; Irizarry, R. A., Hobbs, B., Collin, F., Beazer-Barclay, Y. D., Antonellis, K. J., Scherf, U., et al., Exploration, normalization, and summaries of high density oligonucleotide array probe level data, Biostatistics 2003: 4: 249-64.)

The above method 302 may be employed for a GeneChip with either PM-only probe expression values or the originally intended PM-MM values where an appropriate adjustment 404 is made in either case.

For the PM-only values, global background is preferably estimated according to the technique proposed by Naef et al., which suggests that the approximation of background should be made by taking the collection of all probe-pairs whose IPM-MMI difference is small (in our case, <50). In theory, probes which are affected by only background and noise should have a small IPM-MMI difference, thus those PM probes are more likely to represent the distribution of background. According to this approach, the peak of this distribution is estimated and subtracted as the global background signal. (Naef F, Hacker C R, Patil N, Magnasco M., Empirical characterization of the expression ratio noise structure in high-density oligonucleotide arrays, Genome Biol 2002: 3.)

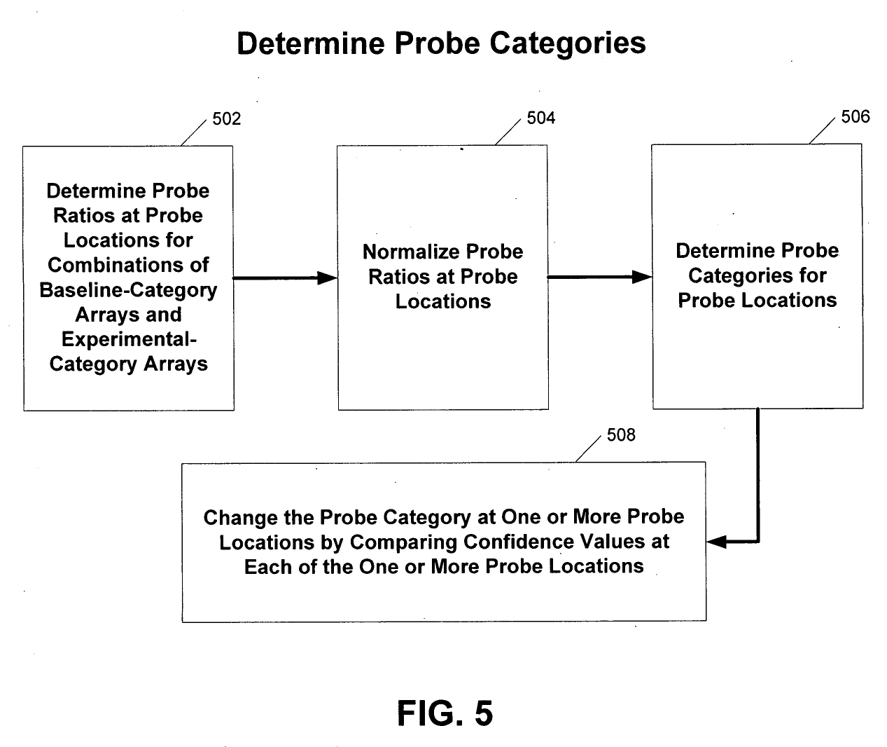

FIG. 5 shows further detail for determining probe categories 306 at the probe locations. First, probe ratios are determined 502 from the probe values at probe locations for combinations of the baseline-category arrays and the experimental-category arrays. For each probe location i=1, . . . ,I, each experimental-category array j=1 . . . m, and each baseline-category array k=1 . . . n, the probe ratios are defined as

$${r}_{ijk} ≡ {log}_{2} \frac{{E}_{ij}}{{B}_{ik}} .$$

Next the probe ratios are normalized 504 at the probe locations. There are several approaches to normalization, ranging from simple averaging to sophisticated quantile estimations. An exemplary normalizing approach is that of Naef et al., which hypothesizes that the distribution of log-ratios should be centered around zero so that summations over probe locations are normalized as:

$${\sum }_{i = 1}^{I} {r}_{ijk} = 0.$$

This assumption is based on the observation that in a pairwise comparison, the vast majority of the ratios in this distribution can be characterized as noise, and in a perfectly normalized system noisy probes are equally likely to be positive as negative. (Naef, F., Hacker, C. R., Patil, N., Magnasco, M. Empirical characterization of the expression ratio noise structure in high-density oligonucleotide arrays. Genome Biol 2002: 3.)

For each two-chip comparison between the experiment group “E” and the baseline group “B,” a ratio value is generated for each probe. The distribution of these transformed ratios in each paired comparison is re-centered to zero by forcing the sum of ratios within a standard deviation of the peak to equal zero. This effectively normalizes the two chips to remove any significant multiplicative bias. That is, the normalized (or new values) are given in terms of the original (or old values) as:

$${r}_{ijk} ≡ {log}_{2} \frac{{E}_{ij}}{{B}_{ik}} - [ {\sum }_{i = 1}^{I} {log}_{2} \frac{{E}_{ij}}{{B}_{ik}} ] / I .$$

Next probe categories for the probe locations are determined 506 from the normalized probe ratios at the probe locations. Each probe i has associated with it a set pᵢ of log-ratios for all each possible combinations of baseline-category arrays and experimental-category arrays:
pᵢ≡{rᵢₗ₁, . . . , rᵢⱼₖ, . . . , rᵢₘₙ}∀j,k

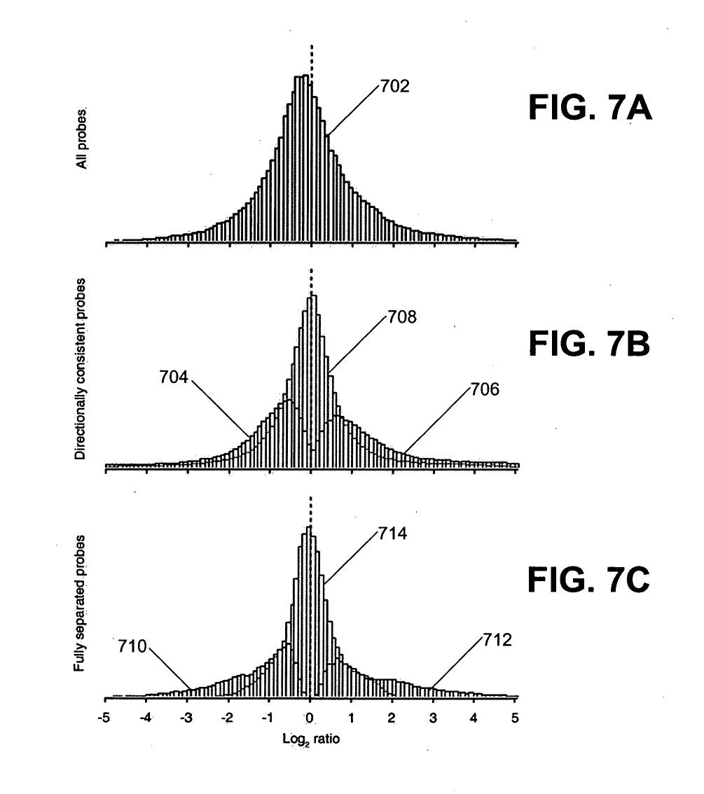

FIG. 7A shows a characteristic distribution 702 of the logged probe ratios from all the possible pair-wise combinations. For the embodiment shown in FIGS. 1-2 where there are one hundred probe locations (I=100), three baseline-category arrays (n=3), and three experimental-category arrays (m=3), there are nine hundred ratio values in the distribution where nine of these ratio values correspond to each probe location (i.e., each log-ratio set pᵢ). Since there is no explicit means for segregating the probes into which group they legitimately belong, as a first approximation the probes are segregated according to directional consistency. Ratios are assigned to three different groups of probes—those that are consistently biased in the baseline direction (S_{B}) or the experimental direction (S_{E}) and those that are indefinite (S_{I}):

$${S}_{E} ≡ \{ {p}_{i} \vert {{⋀}_{j = 1 , k = 1}}^{m , n} {r}_{ijk} > 0 \}$$

$${S}_{B} ≡ \{ {p}_{i} \vert {{⋀}_{j = 1 , k = 1}}^{m , n} {r}_{ijk} < 0 \}$$

$${S}_{I} ≡ \{ {p}_{i} \vert {p}_{i} ∉ {S}_{E} , {p}_{i} ∉ {S}_{B} \}$$

The subsequent distribution is shown in FIG. 7B including distributions for S_{B} 704, S_{E} 706, and S_{I} 708. The probes with ratios demonstrating large changes tend to remain in the same direction; however, at low fold changes the proportion of directionally consistent probes decreases considerably, leading to a “drop” in the distributions.

As a way of refining these distributions of probe ratios, the probe categories are changed 508 at one or more probe locations by determining and comparing confidence values for the baseline category, the experimental category, and the indefinite category at those probe locations.

First, probes in the S_{I} group that statistically demonstrate a change are reassigned to either of the S_{B} group or the S_{E} group. The probability that a probe is a member of a group is calculated by dividing the unequal variance t-test confidence (P(pᵢεS)) by the sum of the t-test confidences from all three groups (P_{Σ}) since the probe must belong in one of the three possible groups. The t-test is well known in the statistical arts for determining a statistical confidence that two data sets come from normal distributions with the same mean value. (Probability and Statistics, M. H. DeGroot and Mark J. Schervish, Addison-Wesley, 2002, p. 502.)

For example, since p<0.05 is the typical standard of biological significance, the following rules for reassigning probe categories are employed with α=0.05:
I. If pᵢεS_{I} and P(pᵢεS_{E})/P_{Σ}>(1−α) then reassign pᵢ to set S_{E}.
II. If pᵢεS_{I} and P(pᵢεS_{B})/P_{Σ}>(1−α) then reassign pᵢ to set S_{B}.

Removal of probes that appear to be biased (or changing) with respect to the baseline and experimental categories leads to an improved estimate of the S_{I} group. Each probe in the biased groups is then compared to this population of probes that are not considered changing. If a probe is not significantly different from the S_{I} group, it is reassigned to that group according to the rule:
III. If pᵢεS_{I} and P(pᵢεS_{I})>α then reassign pᵢ to set S_{I}.

Preferably rules I-III are performed iteratively until group membership stops changing, but a fixed routine (e.g., one iteration) is also possible. Furthermore, alternative statistically based reassignment rules may be employed. The desired result of this reassignment is the isolation of three statistically different collections of probe ratios: two groups (S_{B}, S_{E}) representing targets that demonstrate a significant bias or change in underlying expression, and one group (S_{I}) representing probes whose ratios did not have a statistically convincing bias or change. FIG. 7C shows final distributions for S_{B} 710, S_{E} 712, and S_{I} 714.

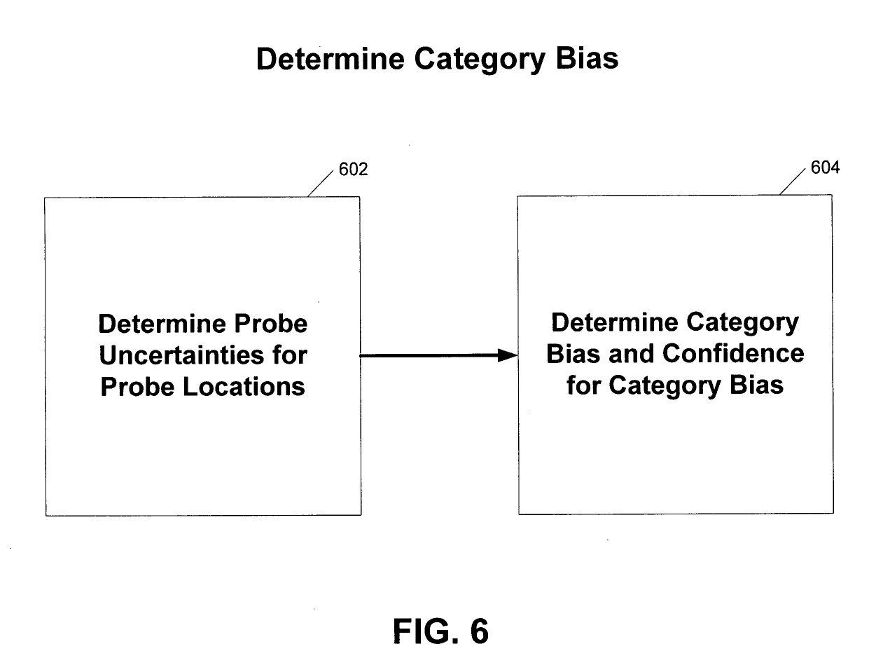

FIG. 6 shows further detail for determining the category bias 308 for the probe group 108. Probe uncertainties are determined 602 for the probe locations in the probe group 108. The probe uncertainty is typically some characterization of the uniformity of the probe ratios at each location. For example, the sample standard deviation of the probe ratio set pᵢ provides a convenient value for the probe uncertainty.

Next a category bias is determined 604 for the probe group 108 together with a corresponding confidence for the category bias. The above-described separation of the individual probe locations into the three groups is performed blindly with respect to probe set membership. The category bias for the probe group 108 is determined 604 by consolidating the calls at the probe locations that make up each probe set. Because of noise and parallel statistics, the probe group 108 will very likely have probes belonging to at least two different category groups (e.g., S_{P} and S_{NC}, S_{N} and S_{NC}, or S_{P}, S_{N} and S_{NC}).

According to one embodiment, determining the category bias 604 includes performing a signed rank test on an ordering of the probe categories according to the probe uncertainties. Signed-rank tests are well-known in the statistical arts for determining a statistical bias between two groups in a data set. (Probability and Statistics, M. H. DeGroot and Mark J. Schervish, Addison-Wesley, 2002, p. 595.)

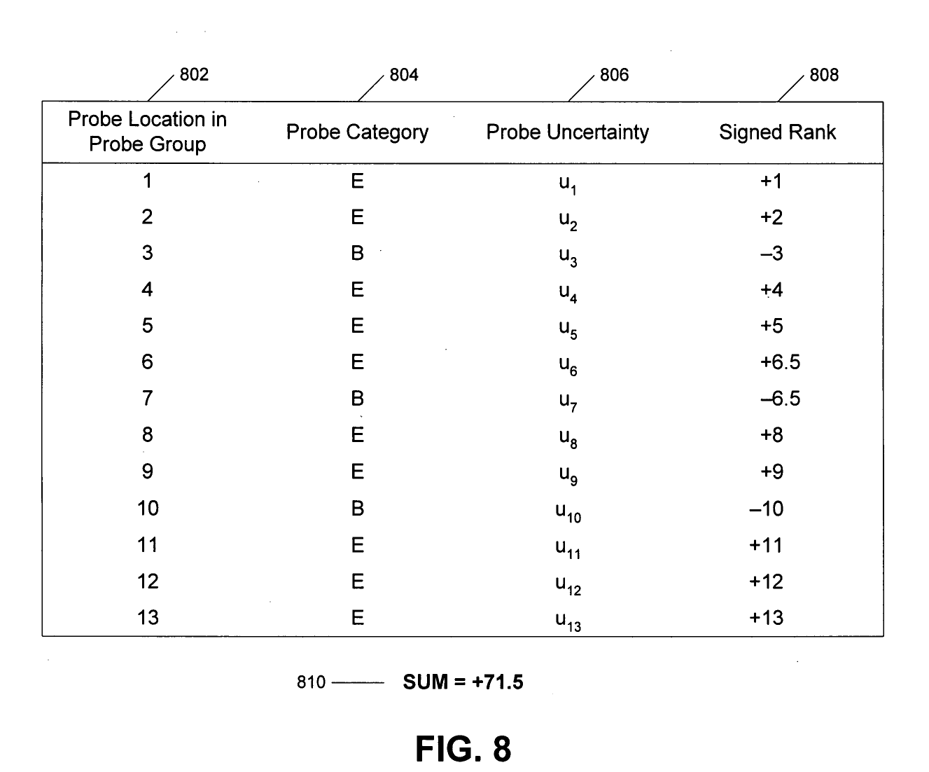

For example, FIG. 8 shows a case where the thirteen probe locations 802 in the probe group 108 have been given a probe category 804 of “B” or “E” (i.e., none of the probes have been given the indefinite category). The probe uncertainties 806 are used to order the entries in the table from highest uncertainty to lowest uncertainty (i.e., u₁≧u₂≧u₃≧ . . . ≧u₁₃). In this context one should note that the probe location entries 802 are arbitrary and that the actual ordering is determined by the probe uncertainties 806 so that the more confident values (i.e., lower probe uncertainties) appear lower in the table. The signed rank values 808 are determined by the ordering of entries determined by the probe uncertainties, where the sixth and seventh values are shown as an average value to illustrate the case of a tie (i.e., u₆=u₇). The signs in this column 808 are determined by the probe categories 804, where the plus sign (for a positive value) is arbitrarily used for the E category and the minus sign (for a negative value) is then used for the B category. Typically the signed rank test is applied to the sum of the positive signed rank values 808 although negative signed rank values 808 could also be used. In this example, the sum 810 of the positive signed rank values (i.e., corresponding to the E category) then provides a category bias (i.e., a preference for the E category) together with an associated confidence. For example, in FIG. 8, one has sum=+71.5, which corresponds to a preference for the E category with a confidence value of approximately 96%.

FIG. 8 shows the case where all the probes are given either the Baseline category or the Experimental category. More generally, some probes may be assigned to the Indefinite category. In this case, there are three possible categories and three possible ways for pair-wise comparisons (e.g., B vs. E, B vs. I, E. vs. I). Then the category bias (and confidence) may reflect a single pair-wise comparison or may reflect a result from all pair-wise comparisons. For example, the comparison of B and E and the comparison of B and I may each show a preference for B, and an overall confidence for this preference may be taken as the minimum (or some average) of the corresponding pair-wise confidences. Alternatively statistical comparison methods may be employed simultaneously over the three possible categories.

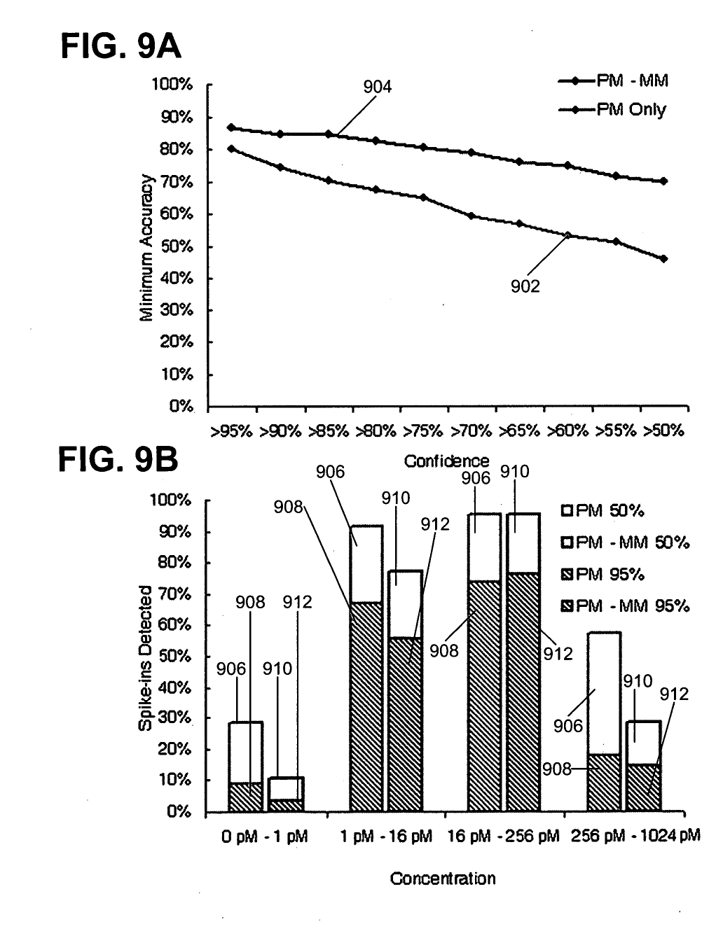

FIGS. 9A-9B illustrate the separation of probes by probe categories for an embodiment of the present invention based on published genomic data. A version of the above-described method 302 was applied to a GeneChip Latin square spike-in data set released by Affymetrix, which has already been used by several techniques including Affymetrix's own MAS 5.0. The spike-in data set was obtained from the Affymetrix website and contains 59 HG-U95 chips. The chips were divided into experimental groups ‘A’-‘T’, with typically three chips in each group. With only a few exceptions, spike-ins followed the Latin square format—each group has thirteen probe sets spiked in at different concentrations from 0.25 pM to 1024 pM with a fourteenth receiving no transcript. The concentrations are shifted from one group to the next, causing most of the spiked probe sets to have a two-fold increase in concentration. Groups ‘M’, ‘N’, ‘O’, and ‘P’ are replicates of one another; as are ‘Q’, ‘R’, ‘S’ and ‘T’. (Hubbell, E., Liu, W. M., Mei, R., Robust estimators for expression analysis, Bioinformatics 2002; 18: 1585-92; Liu, W. M., Mei, R., Di, X., Ryder, T. B., Hubbell, E., Dee, S., et al., Analysis of high density expression microarrays with signed-rank call algorithms, Bioinformatics 2002; 18: 1593-9.)

Since each comparison contains a known number of real changes, this data set is useful in estimating the accuracy of the calls that are made by a method, as well as its general sensitivity. Since the method 302 is preferably performed by comparing two replicate groups at a time, comparisons were run for each possible comparison that provided a two-fold change for most of the comparisons (the smallest fold change possible from this data set)—‘A’ vs ‘B’, ‘B’ vs ‘C’, and so forth. For purpose of this accuracy testing, the extra groups which are replicates of ‘M’ and ‘Q’ were not used. Comparisons were made using both the PM-only and the PM-MM algorithm.

The results were first assessed to determine the number false positives called by the method 302 at different confidence levels. FIG. 9A shows the minimum accuracy as a function of confidence for PM-only data 902 and PM-MM data 904. Here the minimum accuracy is the ratio of the number of correct calls made to the total number of calls made at a given confidence. As described above, the confidence of the category bias of the probe group indicates the confidence that the change seen in the probes is representative of the whole probe set. The confidence statistic is not a probability value and does not imply a probability in the traditional sense. However, the data shown in FIG. 9A suggest that the confidence value correlates very well to the accuracy of each individual call. The correlation is more evident when one considers that several of the changes considered “false positives” are actually probe sets which have the same target sequence as one of the spike-ins. Overall, the PM-MM approach to the analysis returned more accurate results in this setup. However, this use of the MM probe is often criticized in that this probe often contains too much “real” signal, an effect that here works against its ability to bind background similar to the PM probe. In this setup, with the relatively large fold change (i.e., log ratio) of two being the only thing to find, the MM subtraction does well, since if a probe is called a change after the MM subtraction it very likely is a real change.

It is often the case that the minimization of false negatives is more important that the reduction of false positives. Since the spike-ins occurred at various concentrations, it was hypothesized that sensitivity would be concentration dependent. Previous studies have discussed saturation effects occurring at high concentrations, and spike-ins at low concentration are particularly susceptible to becoming lost in global background noise. (Chudin, E., Walker, R., Kosaka, A., Wu, S. X., Rabert, D., Chang, T. K., et al., Assessment of the relationship between signal intensities and transcript concentration for Affymetrix GeneChip arrays, Genome Biol 2002; 3.)

The results were next assessed to determine the number false negatives called by the method 302 at different confidence levels. FIG. 9B shows the percentage of spike-ins detected correctly as a function of concentration for PM-only data at 50% confidence 906 and at 95% confidence 908 and for PM-MM data at 50% confidence 910 and at 95% confidence 912. As expected, the ability of the method 302 to successfully detect changes diminishes at very low and very high concentrations. Not surprisingly, the PM-MM approach also underperformed the PM-only approach. Despite the reduced calls at the extremes, these results demonstrate that at concentrations typical of most mRNA in cells, the method 302 consistently calls existent changes, even at very high stringencies.

A typical advantage of model-based approaches is the down-weighting of probes that are affected by saturation or weakness. The method 302 also weights probes according to reliability, but if too many probes in a probe set are unreliable, as is the case with extreme concentrations, then confidence in any call is diminished.

Additionally (and not illustrated here), as a test of behavior in comparisons with no expected changes, the method 302 was applied to identical comparisons within the experimental groups. The method 302 was applied for every possible paired permutation within ‘M’, ‘N’, ‘O’ and ‘P’ as well as groups ‘Q’, ‘R’, ‘S’, and ‘T’, for a total of twelve comparisons. Since these comparisons are between replicate groups, by definition any genes returned as significant are false positives. The method 302 did not return any probe sets at a confidence higher than 75% and returned very few, even at confidences of as low as 50%.

Importantly, this accurate rejection of probe sets as biased (or changing) occurs despite a relatively large number of individually biased probes. The individual probes are separated by t-test statistics with a probability of p<0.05 in either direction. Therefore, up to 10% of the probes in a replicate comparison such as this could change by pure chance. In these replicate comparisons, between nine thousand and eleven thousand probes were changing significantly in each comparison, but since these changes were due to noise and not to real changes, they did not translate into significant probe set calls. This indicates that while the probe separation provides statistically appropriate results, the probe set consolidation consistently filters out noise due to parallel experimentation and other factors.

Spike-in experiments as described above with reference to FIGS. 9A-9B are useful since there are known concentration differences for a handful of probe sets. However, their general simplicity (consistent fold differences, low systemic variance, etc.) suggests that these experiments are insufficient for a realistic evaluation of the method 302.

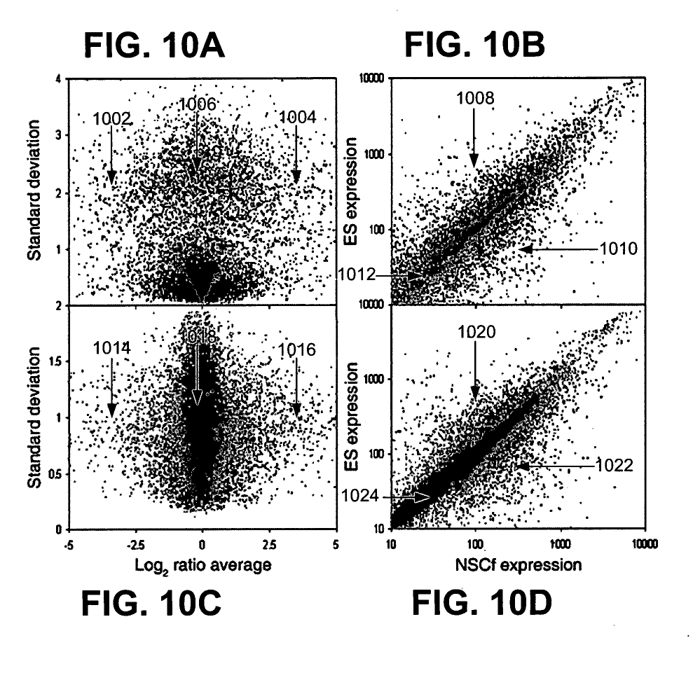

FIGS. 10A-10D illustrate the separation of probes by probe categories for an embodiment of the present invention based on laboratory data. A version of the above-described method 302 was applied to a real data set involving neural and embryonic stem cells. Although “true” changes are unknown in this system, the data set is more typical of real GeneChip experiments, since it has a large variation of unknown changes in different directions as well as replicate groups of different sizes. While the Affymetrix spike-in data set provides an avenue for assessment of accuracy, it is not representative of typical GeneChip experiments. In general, these experiments will result in expression changes in two directions and at a wide range of ratios. Separating real changes from false ones becomes increasingly difficult when genes of interest are changing at low-levels close to experimental noise. In addition, the raw number of genes with differential expression in a real data set can be several orders of magnitude larger than in a spike-in data set. All these differences suggest that the behavior of an analytical method on a highly controlled data set may not necessarily imply desirable performance on a real experimental system.

Therefore, although accuracy cannot be determined explicitly, the above-described method 302 was applied to a published GeneChip experiment. The data set consisted of eight MG-U74Av2 chips in three groups: embryonic stem cells (3 chips-ES), Sox-2 selected fresh neural stem cells (3 chips—NSCf) and cultured neural stem cells (2 chips—NSCc). The experiment was designed to determine the gene expression differences between these three cell types. Since they are cell cultures, considerable difference in expression between cell types was projected and observed in the initial analysis. (D'Amour, K. A., Gage, F. H., Genetic and Functional Differences between Multipotent Neural and Pluripotent Embryonic Stem Cells, Proc Natl Acad Sci USA 2003; 100: 11866-72.)

FIGS. 10A-10D show the results when the method 302 was applied to the ES vs NSCf comparison, in this case using the PM-MM background adjustment, but the results are similar for PM only. FIG. 10A shows a scatter plot of probe locations according to their average fold change (i.e., log ratio average) and probe uncertainty (i.e., standard deviation). Probe locations are separated by their calls as belonging to the ES category 1002, the NSCf category 1004 or the indefinite category 1006. FIG. 10B shows a corresponding scatter plot of probe locations according to their average ES expression and their average NSCf expression. That is, at each probe location the expression values are averaged for each chip category. Again the probe locations are separated by their calls as belonging to the ES category 1008, the NSCf category 1010 or the indefinite category 1012 (i.e., along the diagonal).

As illustrated in FIGS. 10A-10B, the assignment of probe categories is not biased by the average expression level. The probes separate very clearly according to variance and fold change without a significant bias against low expression levels. Probes with a low average fold change are reliably called a change if their variance is low enough, just as a probe showing very high fold changes may be tossed out as noise if its variance is too high.

FIGS. 10C-10D are similar to FIGS. 10A-10B, except that the data plotted corresponds to the consolidated probe groups. FIG. 10C shows a scatter plot of probe groups according to their average fold change (i.e., log ratio average) and probe uncertainty (i.e., standard deviation). Probe groups are separated by their calls as belonging to the ES category 1014, the NSCf category 1016 or the indefinite category 1018. FIG. 10D shows a corresponding scatter plot of probe groups according to their average ES expression and their average NSCf expression. That is, at probe locations in each probe group the expression values are averaged for each chip category. Again the probe groups are separated by their calls as belonging to the ES category 1020, the NSCf category 1022 or the indefinite category 1024 (i.e., along the diagonal).

From examining the results for probe groups in FIGS. 10C-10D, one can see that some low-fold-change probe sets are changing (i.e., biased towards one of the designated categories). However, FIG. 10C shows that since the basic separation is occurring at the probe level, the separation of probe groups is not as well-defined. As illustrated in FIG. 10C, probe sets with similar variance and average fold change are being assigned to different categories. Consistent with the spike-in data results (FIGS. 9A-9B), FIG. 10D shows that relatively few low-intensity probe sets were significantly changing, even though there were numerous changes in low-intensity probes.

One of the strengths of the method 302 is that it low fold change probe sets can reliably be called significant. FIG. 10C shows that the probe consolidation step still allows for very small expression differences to be detected. For example, in the NSCf vs. ES comparison shown, twenty-one probe sets are called changing at 95% confidence despite a fold change of less than 1.2. Small changes can have a large effect if they are involved in critical pathways. In addition, whole tissue samples, in particular nervous tissue, often have very heterogeneous cell populations which can dilute even large changes in this system to levels most methods do not detect. This sensitivity at the low end comes despite stringency at all expression levels—eighteen probe sets were rejected despite apparent 3-fold or higher differences in expression.

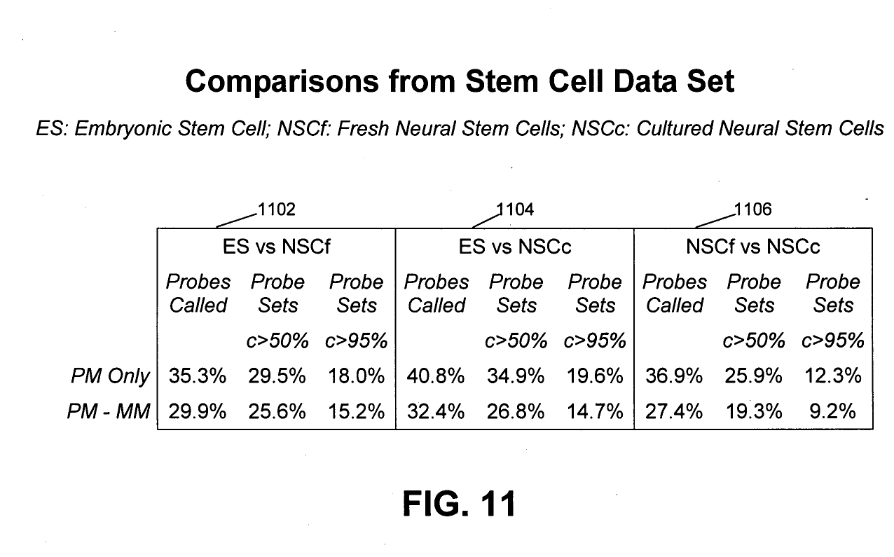

The results shown in FIGS. 10A-10D are consistent with the biological model discussed in the published analysis of this data. FIG. 11 shows summary results for the three comparisons including ES vs. NsCf 1102, ES vs. NSCc 1104, and NSCf vs. NSCc 1106 The comparisons were carried out using both the PM-only approach and the PM-MM approach.

For each of the comparisons 1102, 1104, 1106, the first column of results shows the percentage of probes called to one of the two designated categories (e.g., NSCf or NSCc) and not the indefinite category when the category-changing procedure described above (steps I, II, II with α=0.05) was carried out until the results had substantially converged (e.g., three to four iterations). The second and third columns show the corresponding percentage of probe sets (or probe groups) called to one of the designated categories using the signed rank test with confidences of 50% and 95% respectively. (D'Amour K A, Gage F H., Genetic and Functional Differences between Multipotent Neural and Pluripotent Embryonic Stem Cells, Proc Natl Acad Sci USA 2003; 100: 11866-72.)

Although the results in FIG. 11 show that the PM-only and PM-MM approaches give different results, there is substantial overlap. The PM-only approach returned a larger number of genes as highly significant (confidence >95%) as compared with the full MM subtraction. This difference is probably due to the generally looser nature of the PM-only data that was seen in the spike-in data, and will probably result in fewer false negatives and an increase in false positives. The relatively fewer calls for probe sets in the NSCf vs. NSCc comparison is consistent with the underlying biological similarity for these categories.

Although only certain exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention.

1. A method of determining relative expression for probe groups in probe arrays, comprising:
determining probe values for one or more probe arrays of a baseline category and a plurality of probe arrays of an experimental category, each probe array including a plurality of probes organized by probe locations;
determining, from the probe values corresponding to the probe locations, probe categories for the probe locations, each probe category corresponding to the baseline category, the experimental category or an indefinite category; and
determining, from the probe values and the probe categories, a category bias for a probe group, the probe group including a plurality of the probe locations, and the category bias including a preference for the baseline category, the experimental category or the indefinite category.

2. A method according to claim 1, wherein the one or more probe arrays of the baseline category include a plurality of probe arrays.

3. A method according to claim 1, wherein determining probe values includes:
measuring the probe values from expression levels in the probe arrays; and
adjusting the probe values to remove background signals.

4. A method according to claim 1, wherein determining probe categories for the probe locations includes:
determining probe ratios from the probe values at probe locations for combinations of the baseline-category arrays and the experimental-category arrays;
normalizing the probe ratios at the probe locations; and
determining, from the normalized probe ratios at the probe locations, probe categories for the probe locations.

5. A method according to claim 4, further comprising:
changing the probe categories at one or more probe locations by determining and comparing confidence values for the baseline category, the experimental category, and the indefinite category at the one or more probe locations.

6. A method according to claim 1, wherein determining the category bias for the probe group includes:
determining probe uncertainties for the probe locations in the probe group; and
determining, from the probe categories and probe uncertainties for the probe locations in the probe group, the category bias and a confidence for the category bias.

7. A method according to claim 6, wherein determining the category bias and the confidence for the category bias includes: performing a signed rank test on an ordering of the probe categories according to the probe uncertainties.

8. A method of determining relative expression for probe groups in probe arrays, comprising:
determining probe values for a plurality of probe arrays of a baseline category and a plurality of probe arrays of an experimental category, each probe array including a plurality of probes organized by probe locations;
determining probe ratios from the probe values at probe locations for combinations of the baseline-category arrays and the experimental-category arrays;
normalizing the probe ratios at the probe locations;
determining, from the normalized probe ratios at the probe locations, probe categories for the probe locations, each probe category corresponding to the baseline category, the experimental category or an indefinite category;
changing the probe categories at one or more probe locations by determining and comparing confidence values for the baseline category, the experimental category, and the indefinite category at the one or more probe locations.
determining probe uncertainties for the probe locations in a probe group, the probe group including a plurality of the probe locations; and
determining, from the probe categories and probe uncertainties for the probe locations in the probe group, a category bias for the probe group and a confidence for the category bias, the category bias including a preference for the baseline category, the experimental category or the indefinite category.

9. A method according to claim 8, wherein determining probe values includes:
measuring the probe values from expression levels in the probe arrays; and
adjusting the probe values to remove background signals.

10. A method according to claim 9, wherein determining the category bias and the confidence for the category bias includes: performing a signed rank test on an ordering of the probe categories according to the probe uncertainties.

11. An apparatus for determining relative expression for probe groups in probe arrays, the apparatus comprising executable instructions for:
determining probe values for one or more probe arrays of a baseline category and a plurality of probe arrays of an experimental category, each probe array including a plurality of probes organized by probe locations;
determining, from the probe values corresponding to the probe locations, probe categories for the probe locations, each probe category corresponding to the baseline category, the experimental category or an indefinite category; and
determining, from the probe values and the probe categories, a category bias for a probe group, the probe group including a plurality of the probe locations, and the category bias including a preference for the baseline category, the experimental category or the indefinite category.

12. An apparatus according to claim 11, wherein the one or more probe arrays of the baseline category include a plurality of probe arrays.

13. An apparatus according to claim 11, wherein determining probe values includes:
measuring the probe values from expression levels in the probe arrays; and
adjusting the probe values to remove background signals.

14. An apparatus according to claim 11, wherein determining probe categories for the probe locations includes:
determining probe ratios from the probe values at probe locations for combinations of the baseline-category arrays and the experimental-category arrays;
normalizing the probe ratios at the probe locations; and
determining, from the normalized probe ratios at the probe locations, probe categories for the probe locations.

15. An apparatus according to claim 14, further comprising executable instructions for:
changing the probe categories at one or more probe locations by determining and comparing confidence values for the baseline category, the experimental category, and the indefinite category at the one or more probe locations.

16. An apparatus according to claim 11, wherein determining the category bias for the probe group includes:
determining probe uncertainties for the probe locations in the probe group; and
determining, from the probe categories and probe uncertainties for the probe locations in the probe group, the category bias and a confidence for the category bias.

17. An apparatus according to claim 16, wherein determining the category bias and the confidence for the category bias includes: performing a signed rank test on an ordering of the probe categories according to the probe uncertainties.

18. Computer-readable media tangibly embodying a computer program for determining relative expression for probe groups in probe arrays, the computer program comprising instructions for:
determining probe values for one or more probe arrays of a baseline category and a plurality of probe arrays of an experimental category, each probe array including a plurality of probes organized by probe locations;
determining, from the probe values corresponding to the probe locations, probe categories for the probe locations, each probe category corresponding to the baseline category, the experimental category or an indefinite category; and
determining, from the probe values and the probe categories, a category bias for a probe group, the probe group including a plurality of the probe locations, and the category bias including a preference for the baseline category, the experimental category or the indefinite category.

19. Computer-readable media according to claim 18, wherein the one or more probe arrays of the baseline category include a plurality of probe arrays.

20. Computer-readable media according to claim 18, wherein determining probe values includes:
measuring the probe values from expression levels in the probe arrays; and
adjusting the probe values to remove background signals.

21. Computer-readable media according to claim 18, wherein determining probe categories for the probe locations includes:
determining probe ratios from the probe values at probe locations for combinations of the baseline-category arrays and the experimental-category arrays;
normalizing the probe ratios at the probe locations; and
determining, from the normalized probe ratios at the probe locations, probe categories for the probe locations.

22. Computer-readable media according to claim 21, wherein the computer program further comprises instructions for:
changing the probe categories at one or more probe locations by determining and comparing confidence values for the baseline category, the experimental category, and the indefinite category at the one or more probe locations.

23. Computer-readable media according to claim 18, wherein determining the category bias for the probe group includes:
determining probe uncertainties for the probe locations in the probe group; and
determining, from the probe categories and probe uncertainties for the probe locations in the probe group, the category bias and a confidence for the category bias.

24. Computer-readable media according to claim 23, wherein determining the category bias and the confidence for the category bias includes: performing a signed rank test on an ordering of the probe categories according to the probe uncertainties.
